(19) United States
(12) Patent Application Publication
(10) Pub. No.: US 2007/0012315 A1
(43) Pub. Date: Jan. 18, 2007

(54) Fluid circulation element and spray device which comprises a fluid circulation element

(75) Inventor: Didier Mercier, Aurillac (FR)

Correspondence Address: YOUNG & THOMPSON, 745 SOUTH 23RD STREET, 2ND FLOOR, ARLINGTON, VA, 22202, US

(73) Assignee: QUALIPAC, NEUILL-SUR-SEINE (FR)

(21) Appl. No.: 11/483,705

(22) Filed: Jul. 11, 2006

(30) Foreign Application Priority Data
Jul. 13, 2005 (FR) 05 07530

(51) Int. Cl. A61M 11/00

(52) U.S. Cl. 128/200.14

(57) ABSTRACT

A fluid circulation element includes a nozzle (34) which has a spray hole and a fluid circulation channel between the spray hole and a supply opening, a pushing member (30) which has a recess which is intended to accommodate a pump rod, and a channel which connects the cavity to a supply opening, and a flexible connection element (38) which is connected to the pushing member (30) and the nozzle (34) and which is intended to allow, owing to its flexibility, a relative displacement of the pushing member (30) and the nozzle (34) in the pushing member/nozzle direction and in an activation direction which is substantially perpendicular relative to the pushing member/nozzle direction, the circulation element being formed in one piece from plastics material.

The present invention relates to a fluid circulation element for a spray device, and a spray device which is provided with an element of this type and which is intended for a fluid receptacle.

The invention relates to the field of liquid spraying, in particular the spraying of liquid cosmetic products, for example, for facial or hair care, etcetera.

In this application, a liquid to be sprayed is generally contained in a receptacle which carries a spray device which has a pump which is controlled by a pushing member and which supplies a spray nozzle. The receptacle, when it is used, is held in the hand of the user who directs the spraying towards the area to be treated.

A number of spray devices are already known which generally have the disadvantage of the spray nozzle being displaced in terms of position or rotation when the pushing member is manipulated.

Documents U.S. Pat. No. 6,523,722 and US-2002/008 119 thus both describe spray devices in which a relatively rigid rectilinear tube connects a pump head to a nozzle having a fixed position so that, when the pump head is manipulated by means of a pushing member, the nozzle pivots and thus varies the projection direction of the spraying.

Document U.S. Pat. No. 6,006,958 describes a device in which a nozzle is mounted on a pushing member so that, when the user applies pressure to the pushing member, the nozzle is displaced and brings about a displacement of the spraying direction.

It would be possible to envisage decoupling the nozzle, which is mounted so that it is fixed in terms of position and direction on a receptacle or a cap, from a movable pushing member by connecting them by means of a flexible and non-rigid tube so that the flexibility of the tube prevents the displacement of the pushing member from bringing about a change in direction or position of the nozzle.

It was found that a spray device of this type presented problems in terms of assembly which increased the cost. In particular, it has been found that it was necessary for a return device, which is associated with the receptacle or a cap, to apply to the nozzle a pressure which retains it in an operating position, without impairing the incorporation of the nozzle in the receptacle during production.

The invention relates to the solution of the above-mentioned problems and it relates to producing a fluid spray device so that the receptacle which is provided therewith has no displacement of the nozzle, either in terms of direction or in terms of position, whilst still being able to be produced at a low cost and allowing rapid and inexpensive assembly.

To this end, the invention relates to a fluid circulation element, the whole of which is formed in one piece from plastics material and which comprises a pushing member and nozzle which are connected by means of a flexible connection element, fluid circulation channels of the nozzle and the pushing member being intended to be connected to the circulation element by means of an outer flexible tube.

More precisely, the invention relates to a fluid circulation element for a spray device which comprises a nozzle which has a spray hole and a fluid circulation channel between the spray hole and a supply opening, a pushing member which has a recess which is intended to accommodate a pump rod and a channel which connects the recess to a supply opening, and a flexible connection element which is connected to the pushing member and to the nozzle and which is intended to allow, owing to its flexibility, a relative displacement of the pushing member and the nozzle in the pushing member/nozzle direction, and a relative displacement of the pushing member and the nozzle in an activation direction which is substantially perpendicular relative to the pushing member/nozzle direction.

The fluid circulation element is preferably formed in one piece from plastics material.

In one embodiment, the flexible connection element has a curved arc shape between the pushing member and the nozzle.

In another embodiment, the flexible connection element has a V-shape between the pushing member and the nozzle, the ends of the branches of the V being connected to the pushing member at one side and to the nozzle at the other side. The connection element is preferably narrowed in the proximity of its connection to the pushing member and to the nozzle and/or at the apex of the V.

The channel of the pushing member and the channel of the nozzle preferably both open at the same side, in a direction which is substantially perpendicular relative to the activation direction and which is perpendicular relative to the pushing member/nozzle direction.

Preferably, the flexible connection element further comprises a flexible tube which connects the channels of the pushing member and the nozzle with a U-shape.

The invention also relates to a fluid spray device which comprises a fluid circulation element according to one of the paragraphs above, a pump which is provided with an activation rod which can be moved in an activation direction and a support for the pump and the nozzle of the fluid circulation element. At least a portion of the support is preferably constituted by a liquid receptacle.

The flexible connection element of the circulation element preferably has, in the pushing member/nozzle direction, resilient properties such that, when the pump is mounted on a receptacle and the nozzle is held in a fixed position relative to the receptacle, the flexible connection element applies a force which tends to separate the nozzle from the pushing member.

Further features and advantages of the invention will be better understood from a reading of the following description of embodiments, given with reference to the appended drawings, in which:

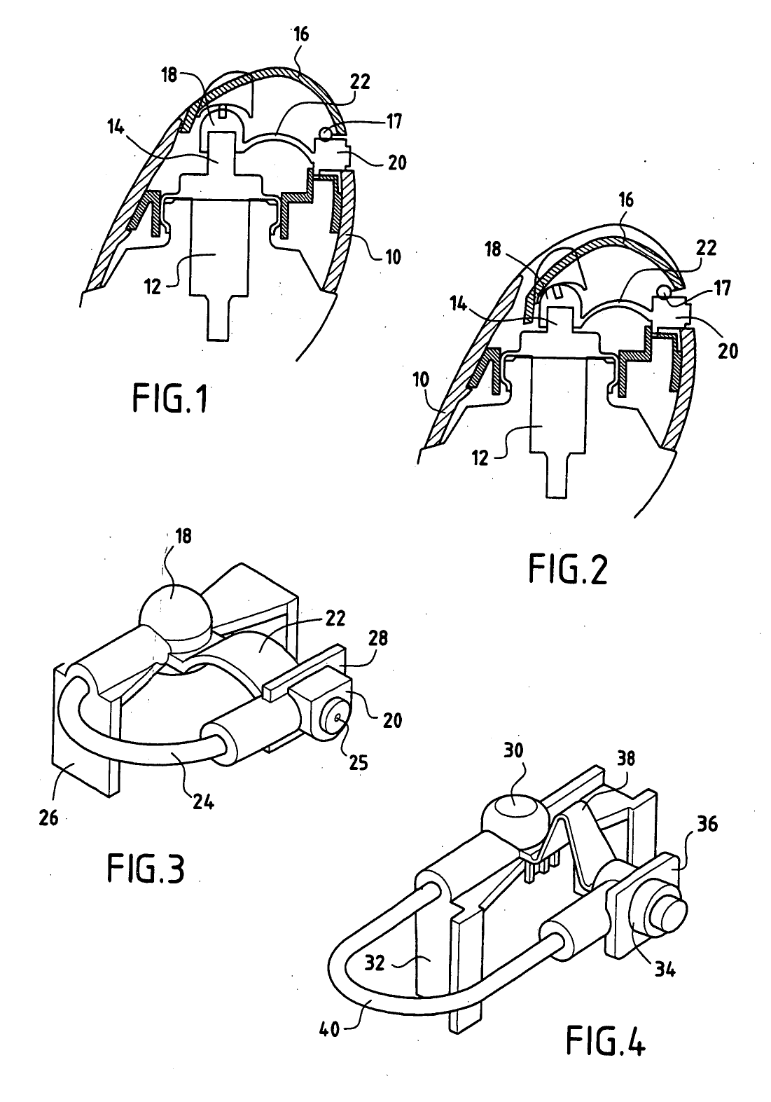

FIG. 1 is a partially sectioned and partially exploded view of a cosmetic fluid receptacle which comprises a spray device according to a first embodiment of the invention, provided with an element for manipulating in the rest position;

FIG. 2 is similar to FIG. 1 but illustrates the manipulation element and the pushing member in an operating position;

FIG. 3 is an enlarged perspective view of the circulation element of the embodiment of FIGS. 1 and 2, provided with a flexible tube;

FIG. 4 illustrates a circulation element according to a second embodiment of the invention, also provided with a flexible tube;

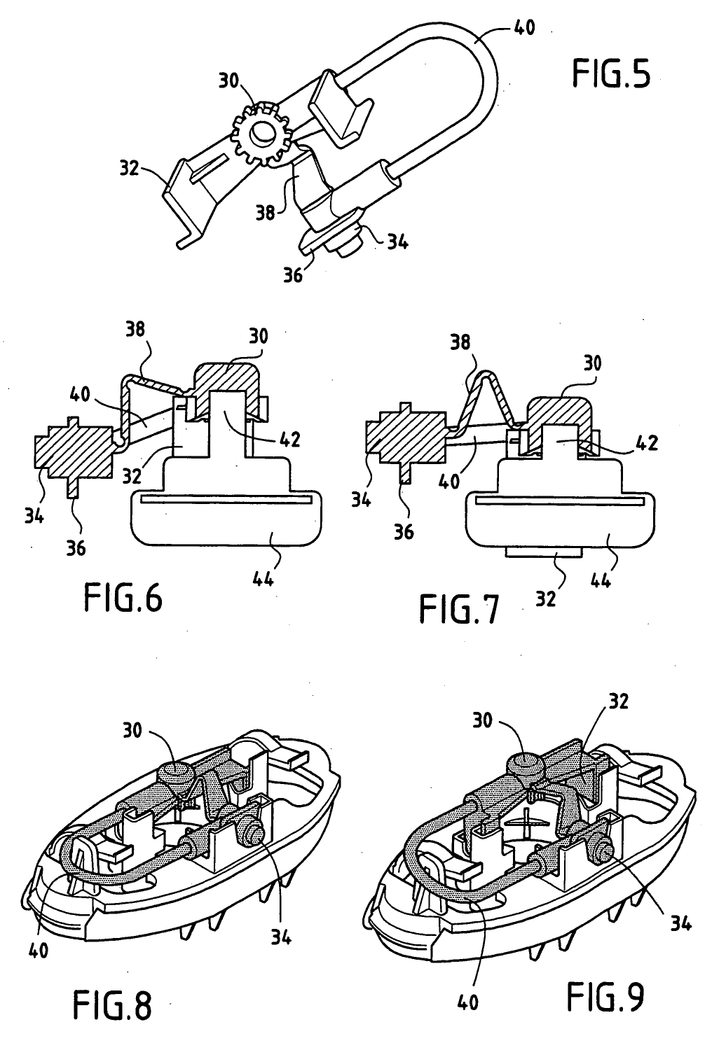

FIG. 5 is a perspective bottom view of the circulation element of FIG. 4;

FIGS. 6 and 7 are schematic and partially sectioned side views illustrating the operation of the element of FIGS. 4 and 5; and

FIGS. 8 and 9 are perspective views which illustrate the element of FIGS. 3 and 4 which is placed in a receptacle head according to the invention, in the two operating and rest positions, respectively.

FIGS. 1 and 2 are partially sectioned and exploded views of the upper portion of a receptacle which is provided with a fluid spray device which comprises the circulation element according to the invention.

The receptacle 10 which can be formed from moulded plastics material supports, using elements which have no specific function in the context of the invention, a pump 12 of known type which is manipulated by means of an activation rod 14. When this rod 14 is pressed in as indicated in FIG. 2, liquid contained in the receptacle 10 is discharged via a central hole of the rod, in known manner.

The receptacle 10 is closed at the upper portion thereof by means of a manipulation element 16 which can be moved by means of pivoting about a shaft 17 so that it displaces a pushing member 18 which is mounted at the end of the activation rod 14. A nozzle 20 has a fixed position relative to the receptacle 10, and is connected to the pushing member 18 by means of a flexible connection element 22.

As shown in FIG. 3, the pushing member 18 and the nozzle 20 are connected by means of a flexible tube 24. More precisely, the liquid conveyed via the hole of the activation rod enters the pushing member 18 which delimits a recess which opens laterally via a supply channel at the end of a support 26 which can be moved in a vertical direction in FIGS. 1 and 2. The channel of the pushing member 18 is connected by means of the flexible tube 24 to an internal supply channel of the nozzle 20, this channel opening via a spray hole 25. It should be noted that the nozzle 20 is provided with a flange 28 which facilitates the assembly thereof on the receptacle.

As shown in FIGS. 1 and 2, the connection element 22, owing to the thinness thereof, allows the pushing member 18 to be displaced in a vertical direction which is the longitudinal direction of the pump 12 and which constitutes an activation direction. On the other hand, owing to the curved shape thereof, the connection element 22 has a level of resilience such that it applies a force which tends to separate the pushing member from the nozzle, when the pushing member is mounted on the activation rod and the nozzle is arranged against the receptacle 10.

According to an advantageous feature of the invention, the pushing member 18, the nozzle 20 and the connection element 22 are formed in one piece from plastics material, preferably by means of injection. The plastics material used may be polyethylene, polypropylene or, more generally, a polyolefine.

Although this feature is not illustrated, the connection element 22 may have two portions which are narrowed in the region of its connection to the pushing member and the nozzle, so that it can apply the resilient force thereof for separating the pushing member and the nozzle without opposing with significant force the displacement of the pushing member in the activation direction.

FIGS. 4 to 9 illustrate a fluid circulation element according to a second embodiment of the invention. The fluid circulation element of this embodiment comprises a pushing member 30 which is carried by a support 32 which facilitates the guiding thereof over its path for activating the pump, a nozzle 34 which is advantageously mounted on a flange 36 for fixing to the receptacle, and a connection element 38 which is formed by a flexible strip in the form of a “V”. The supply channels of the pushing member 30 and the nozzle 34 are connected by means of a flexible tube 40.

FIGS. 4 and 5 clearly indicate the relative arrangement of the elements of the fluid circulation element, and FIGS. 6 and 7 illustrate the operation. In these Figures, the pushing member 30 is placed on the end 42 of the activation rod of a pump 44. Between the rest position of FIG. 6, in which the connection element 38 forms a largely open V-shape, and the position of FIG. 7 in which the connection element 38 forms a much more closed V-shape, only the pushing member is moved in a vertical activation direction by carrying the flexible tube 40 which has pivoted. Conversely, neither the position nor direction of the nozzle has changed.

It should be noted in FIGS. 6 and 7 that the connection element 38 in the form of a strip is advantageously narrowed on the one hand in the proximity of the pushing member 30 and the nozzle 34 and on the other hand at the apex of the V-shape. These optional narrowed portions allow modification of the force which the connection strip 38 applies to the nozzle 34 whilst holding it in position, whilst facilitating the decoupling of the movable pushing member relative to the fixed nozzle.

FIGS. 8 and 9 are general perspective views of the support or receptacle portion which carries the pump (not illustrated), the fluid circulation element, in particular the nozzle 34, and which releases a space for the displacement of the flexible tube 40 and the pushing member 30 whilst guiding the latter by means of its support 32.

In this manner, the fluid circulation element, when it is not provided with the tube 40, constitutes a single piece of plastics material which is moulded, preferably by means of injection moulding. This single component allows the pump rod to be activated, allows spraying and allows the pushing member and the nozzle to be retained in a relative position, by means of a return force being applied which retains the nozzle in position. It facilitates the assembly since the return of the nozzle into its housing is ensured.

1. Fluid circulation element for a spray device, characterised in that it comprises:
a nozzle (20, 34) which has a spray hole and a fluid circulation channel between the spray hole and a supply opening, a pushing member (18, 30) which has a recess which is intended to accommodate a pump rod (14, 42) and a channel which connects the recess to a supply opening, and
a flexible connection element (22, 38) which is connected to the pushing member (18, 30) and to the nozzle (20, 34) and which is intended to allow, owing to its flexibility, a relative displacement of the pushing member (18, 30) and the nozzle (20, 34) in the pushing member/nozzle direction, and a relative displacement of the pushing member (18, 30) and the nozzle (20, 34) in an activation direction which is substantially perpendicular relative to the pushing member/nozzle direction.

2. Circulation element according to claim 1, characterised in that it is formed in one piece from plastics material.

3. Circulation element according to claim 1, characterised in that the flexible connection element (22, 38) has a curved arc shape between the pushing member (18) and the nozzle (20, 34).

4. Circulation element according to claim 1, characterised in that the flexible connection element (22, 38) has a V-shape between the pushing member (30) and the nozzle (34), the ends of the branches of the V being connected to the pushing member (30) at one side and to the nozzle (34) at the other side.

5. Circulation element according to claim 4, characterised in that the flexible connection element (22, 38) is narrowed in the proximity of its connection to the pushing member (30) and to the nozzle (34).

6. Circulation element according to claim 4, characterised in that the flexible connection element (22, 38) is narrowed at the apex of the V.

7. Circulation element according to claim 1, characterised in that the channel of the pushing member (18, 30) and the channel of the nozzle (20, 34) both open at the same side, in a direction which is substantially perpendicular relative to the activation direction and which is perpendicular relative to the pushing member/nozzle direction.

8. Circulation element according to claim 1, characterised in that it further comprises
a flexible tube (24, 40) which connects the channels of the pushing member (18, 30) and the nozzle (20, 34) with a U-shape.

9. Fluid spray device, characterised in that it comprises a fluid circulation element according to claim 1, a pump (12) which is provided with an activation rod (14, 42) which can be moved in an activation direction and a support for the pump (12) and the nozzle (20, 34) of the fluid circulation element.

10. Spray device according to claim 9, characterised in that the flexible connection element (22, 38) of the circulation element has, in the pushing member/nozzle direction, resilient properties such that, when the pump (12) is mounted on a receptacle and the nozzle (20, 34) is held in a fixed position relative to the receptacle, the flexible connection element (22, 38) applies a force which tends to separate the nozzle from the pushing member (18, 30).

11. Circulation element according to claim 2, characterised in that the flexible connection element (22, 38) has a curved arc shape between the pushing member (18) and the nozzle (20, 34).

12. Circulation element according to claim 2, characterised in that the flexible connection element (22, 38) has a V-shape between the pushing member (30) and the nozzle (34), the ends of the branches of the V being connected to the pushing member (30) at one side and to the nozzle (34) at the other side.

13. Circulation element according to claim 12, characterised in that the flexible connection element (22, 38) is narrowed in the proximity of its connection to the pushing member (30) and to the nozzle (34).

14. Circulation element according to claim 5, characterised in that the flexible connection element (22, 38) is narrowed at the apex of the V.

15. Circulation element according to claim 13, characterised in that the flexible connection element (22, 38) is narrowed at the apex of the V.
